(19) United States
(12) Patent Application Publication
(10) Pub. No.: US 2022/0002676 A1
(43) Pub. Date: Jan. 6, 2022

(54) Methods of Preserving Cells for Space Exploration and Compositions Related Thereto

(71) Applicant: Emory University, Atlanta, GA (US)

(72) Inventors: Chunhui Xu, Atlanta, GA (US); Antonio Rampoldi, Atlanta, GA (US); Rajneesh Jha, Bellevue, WA (US)

(21) Appl. No.: 17/344,654

(22) Filed: Jun. 10, 2021

Related U.S. Application Data
(60) Provisional application No. 63/037,194, filed on Jun. 10, 2020.

(51) Int. Cl. C12N 5/077

(52) U.S. Cl. CPC C12N 5/0657; C12N 2501/33; C12N 2500/38; C12N 2501/727; C12N 2500/05; C12N 2500/42; C12N 2500/34; C12N 2500/32

(57) ABSTRACT

This disclosure relates to methods of preserving cells for space exploration, methods of culturing cells, and cell growth media. In certain embodiments, methods comprise contacting cells, such as stem cells, induced pluripotent cells, progenitor cells, and cardiac associated cells, with a cell growth medium disclosed herein providing replicated cells. In certain embodiments, methods comprise preserving and culturing cells in outer space comprising; a) freezing cells providing frozen cells; b) transporting the frozen cells to outer space; c) thawing the cells providing thawed cells; and d) culturing the thawed cells with a growth medium disclosed herein.

### CROSS-REFERENCE TO RELATED APPLICATIONS

This application claims the benefit of U.S. Provisional Application No. 63/037,194 filed Jun. 10, 2020. The entirety of this application is hereby incorporated by reference for all purposes.

### STATEMENT REGARDING FEDERALLY SPONSORED RESEARCH OR DEVELOPMENT

This invention was made with government support under AA025723 and HL136345 awarded by the National Institutes of Health and GA-2017-266 awarded by the Center for the Advancement of Science in Space under NASA Cooperative Agreement No. NNH11CD70A. The government has certain rights in the invention.

### BACKGROUND

Government and private enterprises have projects associated with space exploration. The International Space Station (ISS) is a habitable artificial satellite suited for testing equipment and living organisms for future missions. The long-term effects of an environment with almost no gravity on physiological processes are not entirely understood. For example, Sides et al. report the potential for cardiovascular risks of spaceflight. See Aviat Space Environ Med. 2005, 76(9):877-95. While, Jha et al. report simulated microgravity and 3D culture enhance induction, viability, proliferation, and differentiation of cardiac progenitor cells indicating there may be cardiac benefits to weightless in space. Sci Rep. 2016, 6:30956. Thus, there is a need to develop methods that accurately represent the physiological effects of a low gravity environment on cellular processes in the context of space exploration.

Chen, et al. report culture and osteogenic induction of human mesenchymal stem cells under CO₂-independent conditions. Astrobiology, 2013, 13(4): 370-379.

Battista et al. report development of a carbon dioxide-independent medium, In Vitro Cellular & Developmental Biology, Abstracts, 1991, Vol. 27A, No. 3, pp. 36A-173A.

Vistica et al report a carbon dioxide-independent basal growth medium. Journal of the National Cancer Institute, 1990, 82(12):1055-106.

References cited herein are not an admission of prior art.

### SUMMARY

This disclosure relates to methods of preserving cells for space exploration, methods of culturing cells, and cell growth media. In certain embodiments, methods comprise contacting cells, such as stem cells, induced pluripotent cells, progenitor cells, or cardiac associated cells, with a cell growth medium disclosed herein providing replicated cells. In certain embodiments, methods comprise preserving and culturing cells in outer space comprising; a) freezing cells providing frozen cells; b) transporting the frozen cells to outer space; c) thawing the cells providing thawed cells; and d) culturing the thawed cells with a growth medium disclosed herein.

In certain embodiments, this disclosure relates to cell growth media pH buffered with mono sodium phosphate, dibasic sodium phosphate, beta-glycerophosphate, and sodium bicarbonate for maintaining a pH of near 7.2-7.5 further comprising a) insulin and B27 supplements such as biotin, alpha-tocopherol and alpha-tocopherol acetate ester, vitamin A; b) 4-(2-hydroxyethyl)-1-piperazineethanesulfonic acid (HEPES); c) alanyl-glutamine dipeptide; d) ascorbic acid, ascorbic acid 2-phosphate, or salts thereof; and e) essential amino acids and non-essential amino acids such as glycine, alanine, asparagine, aspartic acid, glutamic acid, proline, serine, or salts thereof.

In certain embodiments, this disclosure relates to cell growth media comprising: a) saccharide or poly saccharide, pyruvate, esters, or salts thereof and a pH buffer with mono sodium phosphate, dibasic sodium phosphate, beta-glycerophosphate, and sodium bicarbonate; b) insulin and B27 supplements such as biotin, vitamin E (e.g., alpha-tocopherol and alpha-tocopherol acetate ester), carnitine, and vitamin A; c) 4-(2-hydroxyethyl)-1-piperazineethanesulfonic acid (HEPES); d) alanyl-glutamine dipeptide; e) ascorbic acid, ascorbic acid 2-phosphate, or salts thereof and f) essential amino acids and non-essential amino acids optionally including glycine, alanine, asparagine, aspartic acid, glutamic acid, proline, serine, or salts thereof. In certain embodiments, the pyruvate salt is sodium pyruvate.

In certain embodiments, the B27 supplements optionally include catalase, transferrin, superoxide dismutase, corticosterone, galactose, ethanolamine, reduced glutathione, carnitine, linoleic acid, linolenic acid, progesterone, putrescine, selenium, triiodothyronine, or salts thereof.

In certain embodiments, the cell growth medium further comprises an antibiotic agent.

In certain embodiments, the vitamin A is retinyl acetate.

In certain embodiments, the osmolarity of the cell growth medium is less than 300, 275, or 250 mOsm.

In certain embodiments, the growth medium comprises serum or a serum supplement.

In certain embodiments, the cell growth medium further comprises eukaryotic cells.

In certain embodiments, the cell growth medium further comprises cardiac cells or cardiac progenitor cells derived from induced pluripotent stem cells.

In certain embodiments, the cell growth medium further comprises an inhibitor of Rho-associated, coiled-coil containing protein kinase (Rock).

In certain embodiments, this disclosure relates to methods of culturing cells comprising contacting cells with a cell growth medium as described herein providing replicated cells.

In certain embodiments, the cells are cultured in the absence carbon dioxide. In certain embodiments, the cells are cultured in the absence of atmospheric carbon dioxide. In certain embodiments, the cells are cultured in the presence of carbon dioxide.

In certain embodiments, the cells are cardiac cells or cardiac progenitor cells derived from induced pluripotent stem cells. In certain embodiments, the cells are cardiac cells or cardiac progenitor cells have a round or spherical shape. In certain embodiments, the cells are cardiac cells or cardiac progenitor cells are in a planar, layered, disk, or sheet shape.

In certain embodiments, this disclosure relates to methods of preserving and culturing cells in outer space comprising; a) freezing cells providing frozen cells; b) transporting the frozen cells to outer space; c) thawing the cells providing thawed cells; and d) culturing the thawed cells with a growth medium as disclosed herein.

In certain embodiments, freezing cells is in a medium containing dimethyl sulfoxide. In certain embodiments, the cells are cardiac progenitor cells rounded or spheroidal in shape suspended in the growth medium. In certain embodiments, the cells comprise 1000 to 2000 cells per spheroidal shape.

### BRIEF DESCRIPTION OF THE SEVERAL VIEWS OF THE DRAWINGS

FIG. 1 shows quantitative data on the differentiation of IMR90 hiPSC-derived 3D cardiac progenitors in a CO₂-independent medium with and without supplements. LIVE/DEAD staining showed viable cells with fluorescence from calcein AM staining and dead cells with fluorescence from ethidium homodimer from spheres. Total number of viable cells (right). Summary for purity of cardiomyocytes (percentage of α-actinin and NKX2.5-positive cells) analyzed by ArrayScan™ (left).

FIG. 2 shows data on the effect of the CO₂-independent medium on the expression of key cardiac genes. 3D cardiac progenitors derived from IMR90 hiPSCs were cultured in either the standard RPMI/B27 medium or the CO₂-independent medium for 7 days. Show is data from qRT-PCR panel indicating relative mRNA expression levels of gene associated with cardiac structure, cardiac hormone, Ca2+ handling proteins, ion channels and cardiac transcription factors (n=3 culture samples; 3 PCR reactions/sample).

FIGS. 3A-3C show data on the effect of the CO₂-independent medium on mitochondrial features of hiPSC-CMs. Differentiated 3D cardiac progenitors derived from IMR90 hiPSCs were cultured in either the standard RPMI/B27 medium or the CO₂-independent medium for 7-14 days.

FIG. 3A shows data derived from representative images of immunocytochemical analysis indicating the expression of mitochondrial import receptor subunit Tom20 by ArrayScan™ (n=5 culture wells; 20 images/well).

FIG. 3B shows data from representative images of mitochondrial membrane potential assay indicating fluorescence intensity of TMRM-positive cells by ArrayScan™ (n=5 culture wells; 20 images/well).

FIG. 3C shows data from qRT-PCR panel indicting relative mRNA expression levels of genes associated with mitochondrial function (n=3 culture samples; 3 PCR reactions/sample).

FIGS. 4A-4B show data on the effect of Rock inhibitor Y-27632 on the survival of cryopreserved 3D cardiac progenitors. Cells derived from IMR90 hiPSCs on differentiation day 6 were used for the formation of cardiac spheres, cryopreserved, and revived in medium with and without the Rock inhibitor into suspension culture. Cultures without cryopreservation were used as a control. Cells on differentiation day 15 were compared with cultures without cryopreservation (control) for morphology, viability, and cardiomyocyte purity.

FIG. 4A shows data on the purity of cardiomyocytes analyzed by ArrayScan™ indicating the percentage of α-actinin and NKX2.5-positive cells (n=5 culture wells; 20 images/well).

FIG. 4B shows data on the total number of viable cells and dead cells recovered from cardiac spheres as analyzed by Trypan blue exclusion (n=3 cultures).

FIG. 5 shows data indicating an effect of pre-incubation prior to cryopreservation on the survival of cryopreserved 3D cardiac progenitors. 3D cardiac progenitors derived from IMR90 hiPSCs were resuspended in cryopreservation medium and pre-incubated at 4° C. before cryopreservation, thawed, and cultured in the CO₂-independent medium for 7 days. Total number of viable cells and dead cells recovered from cardiac spheres were analyzed by trypan blue exclusion (n=3 cultures).

FIGS. 6A-D show data on the effect of supplemental serum on the survival of 3D cardiac progenitors and the function of subsequently derived cardiomyocytes.

FIG. 6A shows data on the total number of viable cells in cultures derived from IMR90 hiPSCs as analyzed by trypan blue exclusion (n=3 cultures).

FIG. 6B shows data on the purity of cardiomyocytes (% NKX2.5 positive cells) in cultures derived from IMR90 hiPSCs analyzed by ArrayScan™ (n=5 culture wells; 20 images/well).

FIG. 6C shows data on the percentage of cells exhibiting regular Ca²⁺ transients or spontaneous Ca²⁺ waves and the representative traces of Ca²⁺ transients in cultures derived from SCVI-273 hiPSCs (n=66-68 single cells).

FIG. 6D shows a summary of Ca²⁺ transient parameters in cultures derived from SCVI-273 hiPSCs (n=64-66 single cells).

FIG. 7 shows a schematic diagram of the experimental design for space flight experiments. SCVI-273 and IMR90 hiPSCs were directed for cardiac differentiation and cardiac progenitors were aggregated into cardiac progenitor spheres on differentiation day 6. The 3D cardiac progenitors were subsequently cryopreserved and sent to the ISS through the SpaceX-20 mission. On the ISS, cardiac progenitor spheres were thawed and cultured in suspension with the CO₂-independent medium in the MVP modules without CO₂ for 22 days.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Prior to describing the various embodiments, the following definitions are provided and should be used unless otherwise indicated.

Use of the term “embodiments” infers that such element(s) are example(s), but not necessarily limited to the example(s).

It must be noted that, as used in the specification and the appended claims, the singular forms “a,” “an,” and “the” include plural referents unless the context clearly dictates otherwise. Thus, for example, reference to “the vitamin A” includes reference to one or more vitamin A isoforms and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Certain of the compounds described herein may contain one or more asymmetric centers and may give rise to enantiomers, diastereomers, and other stereoisomeric forms that can be defined, in terms of absolute stereochemistry at each asymmetric atom, as (R)- or (S)—. The present chemical entities, compositions and methods are meant to include all such possible isomers, including racemic mixtures, tautomer forms, hydrated forms, optically substantially pure forms and intermediate mixtures. In certain embodiments, derivatives are contemplated. In certain embodiments, the compounds may be present in a composition with enantiomeric excess or diastereomeric excess of greater than 60%. In certain embodiments, the compounds may be present in enantiomeric excess or diastereomeric excess of greater than 70%. In certain embodiments, the compounds may be present in enantiomeric excess or diastereomeric excess of greater than 80%. In certain embodiments, the compounds may be present in enantiomeric excess or diastereomeric excess of greater than 90%. In certain embodiments, the compounds may be present in enantiomeric excess or diastereomeric excess of greater than 95%.

As used herein, the term “derivative” refers to a structurally similar compound that retains sufficient functional attributes of the identified analogue. The derivative may be structurally similar because a compound is lacking one or more atoms, substituted with one or more substituents, a salt, in different hydration/oxidation states, e.g., substituting a single for a double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. As used herein, “esters” include alkyl esters of acidic groups, including, but not limited to, carboxylic acids, phosphoric acids, phosphinic acids, sulfonic acids, sulfinic acids, and boronic acids.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, thiol, amino, alkyl, alkoxy, alkanoyl, alkylamino, and alkylthio. “Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, and t-butoxy. “Alkylamino” refers to an alkyl group as defined above attached through an amino bridge. An example of an alkylamino is methylamino, (i.e., —NH—CH₃). “Alkanoyl” refers to an alkyl as defined above attached through a carbonyl bridge (i.e., —(C═O)alkyl). “Alkylthio” refers to an alkyl group as defined above attached through a sulfur bridge. An example of an alkylthio is methylthio, (i.e., —S—CH₃). Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali (basic) or organic salts of acidic residues such as carboxylic acids; and the like. In typical embodiments, the salts are conventional nontoxic acceptable salts including the quaternary ammonium salts, and non-toxic inorganic or organic acids.

### Methods of Culturing Cells and Growth Media

This disclosure relates to methods of preserving cells for space exploration, methods of culturing cells, and cell growth media. In certain embodiments, methods comprise contacting cells, such as stem cells, induced pluripotent cells, progenitor cells, and cardiac associated cells, with a cell growth medium disclosed herein providing replicated cells. In certain embodiments, methods comprise preserving and culturing cells in outer space comprising; a) freezing cells providing frozen cells; b) transporting the frozen cells to outer space; c) thawing the cells providing thawed cells; and d) culturing the thawed cells with a growth medium disclosed herein.

In certain embodiments, this disclosure relates to cell growth media comprising: a) a fuel source, pyruvate, esters, or salts thereof and a pH buffer with mono sodium phosphate, dibasic sodium phosphate, beta-glycerophosphate, and sodium bicarbonate; b) B27 supplements such as biotin, vitamin E, e.g., alpha-tocopherol and alpha-tocopherol acetate ester, and vitamin A; c) 4-(2-hydroxyethyl)-1-piperazineethanesulfonic acid (HEPES); d) alanyl-glutamine dipeptide; e) ascorbic acid, ascorbic acid 2-phosphate, or salts thereof; and f) essential amino acids and non-essential amino acids optionally glycine, alanine, asparagine, aspartic acid, glutamic acid, proline, serine, or salts thereof. In certain embodiments, the pyruvate salt is sodium pyruvate.

In certain embodiments, the cells are cultured in space. In certain embodiments, the cells are cultured in the absence carbon dioxide. In certain embodiments, the cells are cultured in the absence of atmospheric carbon dioxide. In certain embodiments, the cells are cultured in the absence of atmospheric carbon dioxide for more than 10, 15, or 20 days. In certain embodiments, the cells are cultured in the presence of carbon dioxide.

In certain embodiments, the cells are cultured in the presence or low amounts of carbon dioxide. Typical outdoor carbon dioxide concentrations are approximately 400 ppm. In certain embodiments, the cells are cultured in or on a medium exposed to air or gas containing oxygen with less than 300, 200, 100, 50, 10, or 1 ppm of carbon dioxide (at 25° C. and 1 atmosphere). In certain embodiments, the cells are cultured in a medium (liquid, gel, or solid) saturated with oxygen with less than 0.6, 0.3, 0.1, 0.05, 0.01 or 0.001 mg/L of carbon dioxide.

In certain embodiments, the vitamin A is retinyl acetate.

In certain embodiments, the B27 supplements optionally includes catalase, transferrin, superoxide dismutase, corticosterone, galactose, ethanolamine, reduced glutathione, carnitine, linoleic acid, linolenic acid, progesterone, putrescine, selenium, triiodothyronine, salts, or combinations thereof. In certain embodiments, the cell growth medium further comprises an antibiotic agent.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide/disaccharide/polysaccharide, medium chain fatty acids, and/or optionally nucleotides. Typical components in a growth medium include amino acids (histidine, isoleucine, leucine, lysine, methionine, phenylalanine, threonine, tryptophan, valine and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth medium may contain phenol red as a pH indication. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate.

Various growth mediums are known in the art. Minimal Essential Medium (MEM) is a term of art referring to a growth medium that contains calcium chloride, potassium chloride, magnesium sulfate, sodium chloride, sodium phosphate and sodium bicarbonate, essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin M), choline, and inositol (originally known as vitamin B8).

Dulbecco's modified Eagle's medium (DMEM) is a growth medium which contains additional components such as glycine, serine, and ferric nitrate with increased amounts of vitamins, amino acids, and glucose as indicated in Table 1 below.

TABLE 1
Composition of Dulbecco's modified Eagle's medium

| | Concentration |
|---|---|
| Components | (mg/L) |
| Amino Acids | |
| Glycine | 30.0 |
| L-Arginine hydrochloride | 84.0 |
| L-Cystine 2HCl | 63.0 |
| L-Glutamine | 584.0 |
| L-Histidine hydrochloride-H₂O | 42.0 |
| L-Isoleucine | 105.0 |
| L-Leucine | 105.0 |
| L-Lysine hydrochloride | 146.0 |
| L-Methionine | 30.0 |
| L-Phenylalanine | 66.0 |
| L-Serine | 42.0 |
| L-Threonine | 95.0 |
| L-Tryptophan | 16.0 |
| L-Tyrosine disodium salt dihydrate | 104.0 |
| L-Valine | 94.0 |
| Vitamins | |
| Choline chloride | 4.0 |
| Calcium D-pantothenate | 4.0 |
| Folic Acid | 4.0 |
| Niacinamide | 4.0 |
| Pyridoxine hydrochloride | 4.0 |
| Riboflavin | 0.4 |
| Thiamine hydrochloride | 4.0 |
| i-Inositol | 7.2 |
| Inorganic Salts | |
| Calcium Chloride (CaCl₂) (anhyd.) | 200.0 |
| Ferric Nitrate (Fe(NO₃)₃:9H₂O) | 0.1 |
| Magnesium Sulfate (MgSO₄) (anhyd.) | 97.67 |
| Potassium Chloride (KCl) | 400.0 |
| Sodium Bicarbonate (NaHCO₃) | 3700.0 |
| Sodium Chloride (NaCl) | 6400.0 |
| Sodium Phosphate monobasic (NaH₂PO₄—H₂O) | 125.0 |
| Other Components | |
| Phenol Red | 15.0 |

Ham's F-12 medium has high levels of amino acids, vitamins, and other trace elements. Putrescine and linoleic acid are included in the formulation. See Table 2 below.

TABLE 2
Composition of the Ham's F-12 medium

| | Concen- | | Concen- |
|---|---|---|---|
| | tration | | tration |
| Substance | (mg/L) | Substance | (mg/L) |
| NaCI | 7599 | L-methionine | 4.47 |
| KCI | 223.6 | L-phenylalanine | 5 |
| Na₂HPO₄ | 142 | L-proline | 34.5 |
| CaCl₂•2H₂O | 44 | L-serine | 10.5 |
| MgCl₂ | 122 | L-threonine | 12 |
| FeSO₄•7H₂O | 0.834 | L-tryptophan | 2 |
| CuSO₄•5H₂O | 0.00249 | L-tyrosine | 5.4 |
| ZnSO₄•7H₂O | 0.863 | L-valine | 11.7 |
| D-glucose | 1802 | Biotin | 0.0073 |
| Na-pyruvate | 110 | D-Ca-pantothenate | 0.48 |
| Phenol red | 1.2 | Choline chloride | 14 |
| NaHCO3 | 1176 | Folic acid | 1.3 |
| L-alanine | 9 | i-inositol | 18 |
| L-arginine•HCl | 211 | Nicotinic acid amid | 0.037 |
| L-asparagine 13.2 | 13.2 | Pyridoxin•HCI | 0.062 |
| L-aspartic acid | 13.3 | Riboflavin | 0.038 |
| L-cysteine•HCI | 31.5 | Thiamine•HCI | 0.34 |
| L-glutamine | 146 | Vitamin B12 | 1.36 |
| L-glutamic acid | 14.7 | Hypoxanthine | L 4.1 |
| Glycine | 7.5 | Thymidine | 0.73 |
| L-histidine•HCI•H₂O | 21 | Lipoic acid | 0.21 |
| L-isoleucine | 4 | Linoleic acid | 0.084 |
| L-leucine | 13 | Putrescine•2HCI | 0.161 |
| L-lysine•HCI | 36.5 | | |

RPMI 1640 medium contains the reducing agent glutathione and high concentrations of vitamins. RPMI 1640 Medium contains biotin, vitamin B12, and para-aminobenzoic acid (PABA), which are not found in Eagle's Minimal Essential Medium nor Dulbecco's Modified Eagle Medium.

Vistica et al. report in the J Natl Cancer Inst. 1990, 82(12):1055-61 a eukaryotic growth medium developed for CO₂-independent maintenance and propagation of human and nonhuman tumor cell lines. PDRG basal growth medium was supplemented with nucleic acid precursors providing with the following components for culture of leukemia cells: amino acids such as L-alanine, L-arginine (free base), L-asparagine, L-aspartic acid, L-cysteine, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine; the amino acid derivative putrescine; vitamins, coenzymes, and lipids such as biotin, folic acid, nicotinamide, calcium D-pantothenate, choline chloride, pyridoxine, pyridoxal, riboflavin, riboflavin-5′-phosphate, thiamine monophosphate, thiamine, vitamin B12, i-inositol, lipoic acid linoleic acid; carbohydrates and derivatives such as D-glucose, sodium pyruvate; nucleic acid derivatives such as adenosine, cytidine, guanosine, hypoxanthine, inosine, orotic acid, thymidine, uridine; inorganics such as sodium chloride, potassium chloride, dibasic sodium phosphate, monobasic sodium phosphate, magnesium sulfate, magnesium chloride, monobasic potassium phosphate, calcium chloride; inorganic trace elements such as cupric sulfate, ferric nitrate, ferrous sulfate, zinc sulfate; buffers such as sodium bicarbonate and beta glycerophosphate; and phenol red, having a pH=7.3 and final osmolarity=290-295 milliosmoles/kg.

A “B27” supplement is as reported in Brewer et al. Journal of Neuroscience Research 35:567-476 (1993) to contain biotin, L-carnitine, corticosterone, ethanolamine, D(+)-galactose, glutathione (reduced), linoleic acid, linolenic acid, progesterone, putrescine, and retinyl acetate in addition to inorganic salts, e.g., salts of calcium, magnesium, potassium, sodium, and iron, wherein the iron nitrate salts were at about or less than 0.1 mg/L, D-glucose, phenol red, HEPES, sodium pyruvate, proteins, and amino acids. Protein reported include albumin, catalase, insulin, superoxide dismutase, transferrin. Amino acids reported include L-alanine (about or less than 2 mg/L), L-arginine, L-asparagine (about or less than 1 mg/L), L-cysteine (about or less than 1 or 2 mg/L), L-glutamine (absent or including), L-glutamate (absent or at low amounts, e.g., less than 5 micro grams/L), glycine, L-histidine, L-isoleucine, L-leucine, L-lysine, L-methionine, L-phenylalanine, L-proline, L-serine, L-threonine, L-tryptophan, L-tyrosine, and L-valine. The total osmolarity of B27 is about 235 (e.g., 200-250) mOsm which is less than the 335 mOsm of DMEM. In certain embodiments, cell culture medium disclosed herein is contemplated to have osmolarity of less than 300 or 250 mOsm, e.g., between 300-250 mOsm or 200-250 mOsm.

Animal serum such as fetal bovine serum (FBS) is often added to a growth media supplement. Gibco™ KnockOut™ Serum Replacement (KnockOut™ SR) is a more defined formulation designed to directly replace the use of animal serum. Serum replacements typically contain serum proteins such as chromatography purified serum albumin, e.g., lipid-rich albumin which contains albumin protein in complex with lipids, fatty acids, lysophosphatidylcholine, triacylglycerides, phosphatidylcholine, phosphatidic acid, cholesterol, and sphingomyelin; transferrin (iron-saturated); recombinant insulin; amino acids such as glycine, L-histidine, L-isoleucine, L-methionine, L-phenylalanine, L-proline, L-hydroxyproline, L-serine, L-threonine, L-tryptophan, L-tyrosine, L-valine; vitamins and antioxidants such as thiamine, reduced glutathione, ascorbic acid 2-phosphate; and trace elements such as Ag⁺, Al³⁺, Ba²⁺, Cd²⁺, Co₂⁺, Cr³⁺, Ge⁴⁺, Se⁴⁺, Br⁻, I⁻, F⁻, Mn²⁺, Si⁴⁺, V⁵⁺, Mo⁶⁺, Ni²⁺, Rb⁺, Sn²⁺, Zr⁴⁺. These supplements are intended to remove or reduce in serum (so that the medium does not contain or contains consistently low concentrations of) growth factors, steroid hormones, glucocorticoids, cell adhesion factors, immunoglobulins, and mitogens, concentrations of which can vary in the serum of individual animals.

Matrigel™ matrix is a solubilized basement membrane preparation extracted from the Engelbreth-Holm-Swarm (EHS) mouse sarcoma, a tumor rich in extracellular matrix proteins. Matrigel™ a partially defined extracellular matrix (ECM) extract including laminin (a major component), collagen IV, heparan sulfate proteoglycans, entactin/nidogen, and a number of growth factors such as TGF-beta 1, epidermal growth factor, insulin-like growth factor, fibroblast growth factor, tissue plasminogen activator. Braam et al. report that in Matrigel™ or natural and recombinant vitronectin was effective in supporting sustained self-renewal and pluripotency in three independent human embryonic stem cells lines, Stem Cells, 2008, 26(9):2257-65.

In certain embodiments, this disclosure relates to methods of preserving and culturing cells in outer space comprising; a) freezing cells providing frozen cells; b) transporting the frozen cells to outer space; c) thawing the cells providing thawed cells; and d) culturing the thawed cells with a growth medium as disclosed herein.

In certain embodiments, freezing cells is in a cryoprotectant medium containing dimethyl sulfoxide. CryoSOfree™ is the animal protein free cryoprotectant containing polyampholytes with an appropriate ratio of amino and carboxyl groups (COOH to amine ration between 0.4 to 0.8). In certain embodiments, the cells are cardiac progenitor cells rounded or spheroidal in shape suspended in the growth medium. In certain embodiments, the cells comprise 1000 to 2000 cells per spheroidal shape. In certain embodiments, the cells are pre-incubated for a duration at 1, 2, 3, 4, 5, 6, 7, 8, 9, or 10° C. before being frozen. In certain embodiments, the duration is for at least 5, 10, 15, 20, 25, 30, 35, 40, 45, or 50 minutes. In certain embodiments, the cells are frozen by exposure to liquid carbon dioxide or liquid nitrogen.

“Carnitine” is also known as 3-hydroxy-4-(trimethylazaniumyl)butanoate. Carnitine facilitates the using fatty acids as a source of energy. Carnitine is converted to acetyl-carnitine and propionyl-carnitine. As used herein, the term a “carnitine” includes carnitine and “carnitine analogs” and encompasses racemic or essentially pure L-carnitine (carnitine), or a corresponding alkanoyl-carnitine such as acetyl-carnitine or propionyl-carnitine, or a suitable salt of such compounds such as L-carnitine tartrate, L-carnitine fumarate, L-carnitine-magnesium-citrate, acetyl-L-carnitine tartrate, acetyl-L-carnitine-magnesium-citrate, or any mixture of the aforementioned compounds.

In certain embodiments, a growth medium disclosed herein comprises other vitamins such as vitamin A, vitamin D, vitamin E, or combinations thereof. As used herein, a “vitamin A” refers to a group of fat-soluble retinoids, including retinol, retinal, and retinyl esters, retinyl acetate, retinyl palmitate and provitamin A carotenoids, alpha-carotene, beta-carotene, gamma-carotene, and beta-cryptoxanthin. As used herein, a “vitamin D” refers to a family of fat-soluble steroids derived from 7-dehydrocholesterol, ergosterol and 7-dehydrositosterol. Examples include cholecalciferol ergocalciferol, lumisterol, sitocalciferol, and 22-dihydroergocalciferol. As used herein, a “vitamin E” refers to a family of molecules having a chromanol ring (chroman ring with an alcoholic hydroxyl group) and a 12-carbon aliphatic side chain containing two methyl groups in the middle and two more methyl groups at the end. The side chain of the tocopherols is saturated, while the side chain of the tocotrienols contain three double-bonds, which adjoin a methyl group. The tocopherols and the tocotrienols exist in four isoforms, referred to as alpha, beta, gamma, and delta isoforms. The isoforms are named on the basis of the number and position of the methyl groups on the chromanol ring. The alpha form has three methyl groups, the beta and gamma forms have two methyl groups and the delta for has only one methyl group. A “vitamin E” may be alpha-tocopherol, beta-tocopherol, gamma-tocopherol, alpha-tocotrienol, beta-tocotrienol, and gamma-tocotrienol. A “vitamin E” also includes esters of a vitamin E isoform. For example, a “vitamin E” includes esters of a tocopherol, including acetates and succinates.

In certain embodiments, the cell growth medium further comprises an inhibitor of Rho-associated, coiled-coil containing protein kinase (Rock). In certain embodiments, the inhibitor of Rho-associated, coiled-coil containing protein kinase (Rock) is 4-[1-aminoethyl]-N-4-pyridinylcyclohexanecarboxamide (Y-27632), 6-chloro-N4-(3,5-difluoro-4-((3-methyl-1H-pyrrolo[2,3-b]pyridin-4-yl)oxy)phenyl)pyrimidine-2,4-diamine (azaindole), 1-(3-hydroxybenzyl)-3-(4-(pyridin-4-yl)thiazol-2-yl)urea [RKI-1447], N-(6-fluoro-1H-indazol-5-yl)-2-methyl-6-oxo-4-(4-(trifluoromethyl)phenyl)-1,4,5,6-tetrahydro pyridine-3-carboxamide (GSK429286A), 5-((1,4-diazepan-1-yl)sulfonyl)isoquinolin-1(2H)-one (hydroxyfasudil), 5-((1,4-diazepan-1-yl)sulfonyl)isoquinoline (fasudil), 4-fluoro-5-((2-methyl-1,4-diazepan-1-yl)sulfonyl)isoquinoline (ripasudil), or 4-(3-amino-1-(isoquinolin-6-ylamino)-1-oxopropan-2-yl)benzyl 2,4-dimethylbenzoate (netarsudil) or salts thereof.

### Cells for Use in Culture

In certain embodiments, a cell growth medium disclosed herein further comprises a eukaryotic cell. In certain embodiments, the cell growth medium further comprises cardiac cells or cardiac progenitor cells derived from induced pluripotent stem cells. In certain embodiments, the cells are cardiac cells or cardiac progenitor cells derived from induced pluripotent stem cells. In certain embodiments, the cells are cardiac cells or cardiac progenitor cells having a round or spherical shape. In certain embodiments, the cells are cardiac cells or cardiac progenitor cells having in a planar, layered, disk, or sheet shape.

In certain embodiments, this disclosure relates to methods of culturing cells comprising contacting cells with a cell growth medium as described herein providing replicated cells. In certain embodiments, the cells may be stem cells such as embryonic stem cells, pluripotent stem cells, or induced pluripotent stem cells.

Embryonic stem cells (ESCs) originate from the inner cell mass of mammalian blastocysts which occur 5-7 days after fertilization. ESCs remain undifferentiated indefinitely under defined conditions and differentiate into so-called embryonic bodies when cultivated in vitro. Having pluripotency, they are capable of differentiating into all cell types. Adult stem cells (somatic cells), such as hematopoietic, neural, and mesenchymal stem cells have an ability to become more than one cell type but do not have the ability to become any cell type.

Induced pluripotent stem cells (iPSCs) are differentiated cells reprogrammed to return to a pluripotent state. Reprogrammed differentiated cells may be accomplished using genes involved in the maintenance of ESC pluripotency, e.g., Oct3/4, Sox2, c-Myc, Klf4, and combinations thereof. The term “induced pluripotent stem cells” refers to cells that are reprogrammed from somatic or adult stems cells to an embryonic stem cell (ESC)-like pluripotent state. See Takahashi et al. “Induction of pluripotent stem cells from mouse embryonic and adult fibroblast cultures by defined factors,” Cell, 2006, 126(4):663-676. Park et al. report reprogramming of human somatic cells to pluripotency with defined factors, Nature, 2008, 451(7175):141-146. Thus, making iPSCs from cells can typically be accomplished by in trans expression of OCT4, SOX2, KLF4 and c-MYC. Colonies appear and resemble ESCs morphologically. Alternatively, certain multipotent stem cells may require less than all of the four transcripts, e.g., cord blood CD133+ cells require only OCT4 and SOX2 to generate iPSCs. For additional guidance in generating iPSCs, see Gonzalez et al. “Methods of making induced pluripotent stem cells: reprogramming a la carte,” Nature Reviews Genetics, 2011, 12:231-242.

Induced pluripotent stem cells typically express alkaline phosphatase, Oct 4, Sox2, Nanog, and/or other pluripotency-promoting factors. It is not intended that induced pluripotent stem cells be entirely identical to embryonic cells. Induced pluripotent stem cells may not necessarily be capable of differentiating into any type of cell.

In certain embodiments, this disclosure contemplates that induced pluripotent stem cells are derived from adult stem cells or mesenchymal stem cells. These terms include the cultured (self-renewed) progeny of cell populations. The term “mesenchymal stromal cells” or “mesenchymal stem cells” refers to the subpopulation of fibroblast or fibroblast-like nonhematopoietic cells with properties of plastic adherence and capable of in vitro differentiation into cells of mesodermal origin which may be derived from bone marrow, adipose tissue, Wharton's jelly in umbilical cord, umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells.

In certain embodiments, this disclosure contemplates that induced pluripotent stem cells are derived from human adipose stem cells. Sun et al. Proc Natl Acad Sci USA., 2009, 106(37):15720-15725 report induced pluripotent stem (iPS) cells can be generated from adult human adipose stem cells (hASCs) freshly isolated from patients.

In certain embodiments, this disclosure contemplates that induced pluripotent stem cells are derived bone marrow derived mesenchymal stromal cells. Bone marrow derived mesenchymal stromal cells are typically expanded ex vivo from bone marrow aspirates to confluence. Certain mesenchymal stromal/stem cells (MSCs) share a similar set of core markers and properties. Certain mesenchymal stromal/stem cells (MSCs) may be defined as positive for CD105, CD73, and CD90 and negative for CD45, CD34, CD14 or CD11b, CD79α or CD19, and HLA-DR surface markers, and have the ability to adhere to plastic. See Dominici et al. “Minimal criteria for defining multipotent mesenchymal stromal cells,” The International Society for Cellular Therapy position statement, Cytotherapy, 2006, 8(4):315-317.

Kattman et al. report multipotent flk-1+ cardiovascular progenitor cells give rise to the cardiomyocyte, endothelial, and vascular smooth muscle lineages. Dev Cell. 2006; 11(5):723-73. Lindsley et al. report Mesp1 coordinately regulates cardiovascular fate restriction and epithelial-mesenchymal transition in differentiating ESCs. Cell Stem Cell. 2008. Bondue et al. report Mesp1 acts as a master regulator of multipotent cardiovascular progenitor specification promoting the stable expression of cardiomyogenic transcription factors, including Nkx2.5, Gata4, Isl1, and myocardin, in a cell-autonomous manner. Cell Stem Cell. 2008; 3(1):69-84.

### Cryopreservation and CO₂-Independent Culture of 3D Cardiac Progenitors for Spaceflight Experiments

Space experimentation of cardiomyocyte differentiation from human induced pluripotent stem cells offers an exciting opportunity to explore the potential of these cells for disease modeling, drug discovery and regenerative medicine. Previous studies on the International Space Station were done with 2D non-cryopreserved cultures of cardiomyocytes being loaded and cultivated in spaceflight culture modules with carbon dioxide (CO₂). Methods of cryopreservation and CO₂-independent culture of 3D cardiac progenitors are reported herein. Cryopreservation allows preparation and pretesting of the cells before spaceflight, making it easier to transport the cell culture, reducing the impact of strong gravitational force exerted on the cells during the launch of spaceflight, and accommodating a more flexible working schedule for the astronauts. The use of CO₂-independent medium with supplements supports cell growth and differentiation without a CO₂ incubator. A spaceflight experiment was conducted through the SpaceX-20 mission to evaluate the effect of microgravity on the survival and differentiation of 3D cardiac progenitors. Cryopreserved cardiac progenitor spheres were successfully cultivated in a spaceflight culture module without CO₂ for 3 weeks aboard the International Space Station. Beating cardiomyocytes were generated and returned to the earth for further study.

Cryopreserved spheres were successfully thawed and further cultivated for 3 weeks on board the ISS. Initial results showed healthy and beating cardiomyocytes generated from the progenitor cells similar to those observe at the end of the cardiomyocyte differentiation on the ground. With modifications in medium composition, these protocols might be adapted for other cell types as well.

The cell preparation, cell culture conditions on the ISS, culture format and cell types differ compared with other reports. Previous studies on the ISS were done with non-cryopreserved cultures being loaded and cultivated in self-contained modules with CO₂. Here, cryopreserved cells were sent to the ISS, thawed, and cultured by the astronauts on the ISS in a CO₂-independent condition. The cells were cultured cells in a 3D suspension culture format instead of the 2D format. The starting cells were cardiac progenitors or early-stage immature cardiomyocytes derived from hiPSCs whereas previous studies used cardiovascular progenitors derived from human heart tissues and late-stage hiPSC-CMs.

A CO₂-independent culture of 3D cardiac progenitors is a prerequisite for a cell culture module that does not provide CO₂. A CO₂-independent culture is also highly desirable for this and other types of cells that are to be experimented on the ISS. Without the need of CO₂ supply, a CO₂ tank would not need to be carried on a spaceflight and thus precious space/mass allowances can be devoted for other purposes. In addition, these new techniques facilitate practical operations of stem cell-cardiomyocytes for ground-based applications such as live-cell shipping and live-cell imaging for long durations where CO₂ supply is limited.

The CO₂-independent culture medium used not only supports the long-term survival but also the differentiation of cardiac progenitors. Based on cell viability and cardiomyocyte purity, differentiation of the 3D cardiac progenitors in the CO₂-independent medium in an incubator without CO₂ is comparable to those cultured in conventional medium with CO₂.

Cardiac spheres can be efficiently frozen and thawed with minimal loss of cell viability. While single cells from cultures could be frozen at −80° C. immediately after being resuspended in freezing medium, cardiac spheres were a pre-incubation of 20-30 min at 4° C. for the best outcome. Pre-incubation of the spheres with cryopreservation medium can increase the survival of the cardiac progenitors by several fold.

The cryopreservation protocol provides advantages for pre-flight, launch and on-orbit operations. It allows enough time to prepare the cells in advance, which is important as spaceflight launch can be delayed for various reasons. It also offers the possibility to pre-test aliquots of cardiac progenitor cell preparations to ensure that they can be efficiently differentiated into cardiomyocytes in standard conditions before being sent to the ISS; this is important as differentiation outcomes can vary from batch to batch; therefore cell preparations of the highest quality (as well as optimum culture condition) should be used given limited opportunities of the spaceflight experiments and the costly nature of spaceflight missions. In addition, cryopreserved samples can avoid the aggregation of spheres during spaceflight launch that would otherwise occur for suspension samples. In addition, cryopreservation will also accommodate a more flexible working schedule for the astronauts to prepare the modules and thaw the cells when it best suits their timetable onboard the ISS.

Furthermore, cryopreservation of cardiac spheres also has a broad impact for the application of hiPSC-CMs in regenerative medicine, given significant advantages of transplantation of cryopreserved cells in pre-clinical and clinical settings. Cryopreservation is preferable for cell therapy since it allows enough time for pre-testing the quality of cell preparations prior to transplantation and confirmation of sufficient quantity of cells for transplantation. It will also facilitate transportation of the cells to operation sites.

### Spaceflight Experiment

Three-dimensional (3D) spheres of cardiac progenitors were generated in Aggrewell™ 400 plates on differentiation day 6 and cryopreserved on day 7. The cryopreserved samples were sent to the ISS through the SpaceX-20 mission, a mission launched by the aerospace company SpaceX on Mar. 6, 2020 for the delivery of cargo and supplies to the ISS. Following culture for 22 days on the ISS, cell culture samples were returned to the ground via warm storage. Upon arrival at Emory University, cardiac spheres were transferred immediately to an incubator and allowed to recover overnight. The following day cardiac spheres were transferred from the collection bags into low adhesion dishes. Medium was changed from the CO₂-independent medium to standard cardiomyocyte culture medium RPMI/B27. Cardiac spheres were then maintained in a standard incubator.

Differentiation of Cardiac Progenitors without CO₂ Supply

To prepare experiments on the ISS with the cell culture hardware that does not have the supply of CO₂, 3D cardiac progenitor cells were prepared. Culture conditions were evaluated for the ability to support cell survival and differentiation of cardiac progenitors without CO₂. Initially 3D cardiac progenitors were directly cultured in the standard RPMI/B27 medium supplemented with additional components (including buffering agent HEPES, ascorbic acid, non-essential amino acids, and GlutaMAX™) to examine if this formulation could support the differentiation of hiPSCs at 37° C. without CO₂. However, cells did not look healthy after 5 days. Spheres cultured in an incubator with CO₂ survived well, whereas the spheres without CO₂ appeared loosely aggregated with apoptotic/necrotic cells floating in the medium. The color of the medium in the two sets of culture also appeared strikingly different: in the standard culture the medium turned yellow which was normal in routine culture, but in the culture without CO₂ it was visibly pink as the medium was not utilized by cells.

Experiments were performed to determine whether a commercially available basal CO₂-independent medium could support the growth and differentiation of hiPSCs. The commercial CO₂-independent medium is a non-HEPES proprietary medium that was formulated for transporting cells or tissue under atmospheric conditions without CO₂ supply. The CO₂-independent medium was supplemented with other components described in Table 3 in addition to B27, including HEPES, ascorbic acid, GlutaMAX™ and non-essential amino acids.

TABLE 3
Composition of CO₂-independent medium for cardiac
progenitors to differentiate into cardiomyocytes.

| | | Volume | |
|---|---|---|---|
| Ingredients | Company Cat. No. | (100 mL) | |
| base CO₂-independent medium | Gibco 18, 045-088 | Make up | |
| | | to 100 mL | |
| B27 supplement with Insulin (50X) | Gibco 17, 504-044 | 2 | mL |
| FBS | Hyclone SH30396-03 | 3 | mL |
| HEPES (1M) | Gibco 15, 630-080 | 1.5 | mL |
| GlutaMAX-I (100X) | Gibco 35, 050-061 | 1 | mL |
| 1-ascorbic acid 2-phosphate | Sigma A8960-5G | 25 | μL |
| sesquimagnesium salt hydrate | | | |
| (100 mg/mL, stock) | | | |
| non-essential amino acids (100X) | Gibco 11, 140-050 | 1 | mL |
| penicillin/streptomycin (100X) | Gibco 15, 140-122 | 1 | mL |

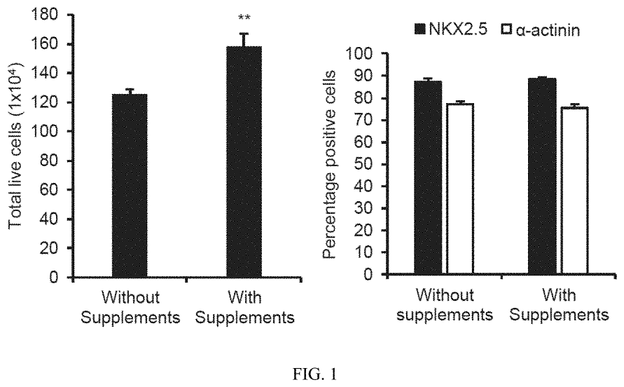

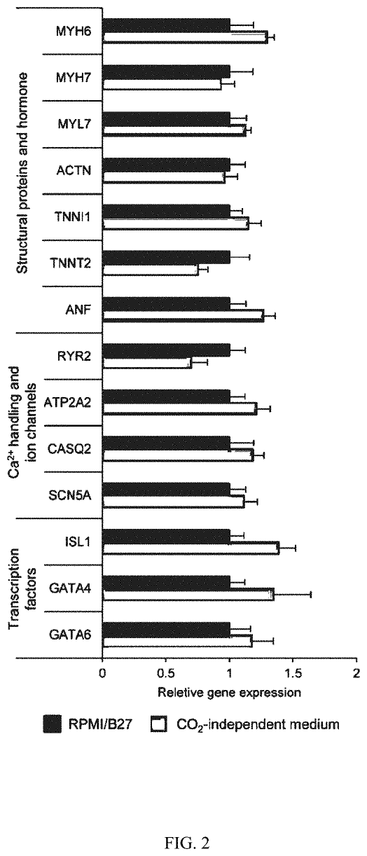

Three-dimensional (3D) cardiomyocyte differentiation was performed to compare the CO₂-independent medium without the supply of CO₂ with normal culture condition (RPMI/B27 medium in a CO₂ incubator). 3D cardiac progenitors were switched to the CO₂-independent medium containing the supplements (Table 1). Control cardiac progenitor spheres in RPMI/B27 were kept in conventional culture conditions under 5% CO₂ supply. Cells in both culture conditions were observed daily and did not show differences in morphological appearances before day 20 when the cells were harvested. The typical beating of cardiomyocytes was observed as early as day 9 in both the culture conditions and the cells continued beating until harvesting. LIVE/DEAD staining showed that the cell viability was similar in the culture grown in the CO₂-independent medium in an incubator without CO₂ and the culture grown in regular RPMI/B27 medium in an incubator with CO₂. The diameters of the spheres in these conditions were also similar. High-throughput ArrayScan™ analysis of NKX2.5 showed that the purity of hiPSC-CMs was comparable between these conditions. In addition, the supplements added to the CO₂-independent medium increased the viable cell counts compared with the medium without the supplements (FIG. 1). These results indicate that long-term cell survival and differentiation of cardiac spheres can be supported without CO₂ supply using the CO₂-independent medium.

Characterization of hiPSC-CMs from Cardiac Progenitors Maintained in the CO₂-Independent Medium

The sarcomeric structures and gene expression of the hiPSC-CMs derived from cardiac progenitors maintained in the CO₂-independent medium were examined. Sarcomeric structures within cardiomyocytes were similarly developed in both RPMI/B27 medium and the CO₂-independent medium as detected by immunostaining using antibodies against α-actinin, a protein in the sarcomeric Z line. Compared with the cells maintained in the standard medium RPMI/B27, cells maintained in the CO₂ independent medium expressed similar levels of genes related to cardiac structure (MYH6, MYH7, MYL7, ACTN1, TNNI1, and TNNT2), cardiac hormone (ANF), Ca2+ handling proteins and ion channels (RYR2, ATP2A2, CASQ2, and SCN5A), and cardiac transcription factors (ISL1, GATA4, and GATA6).

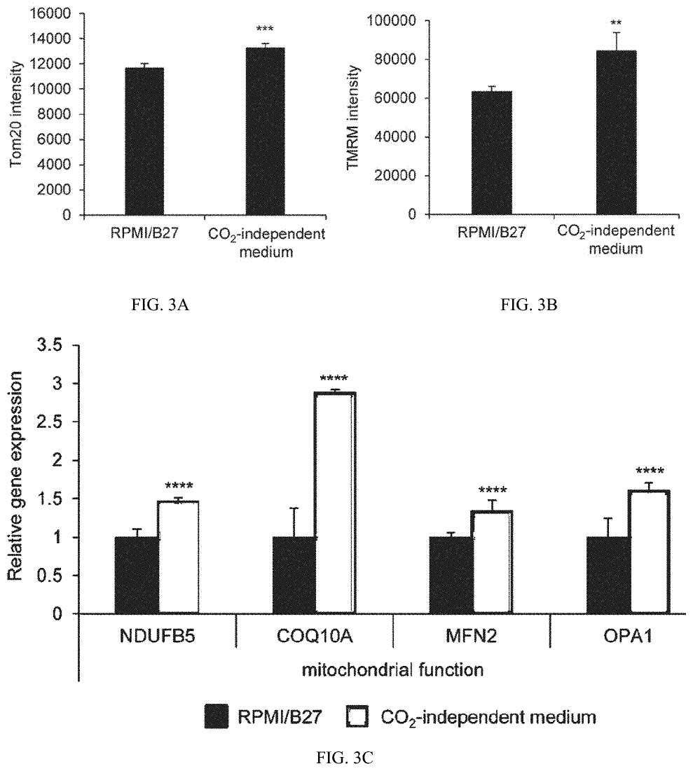

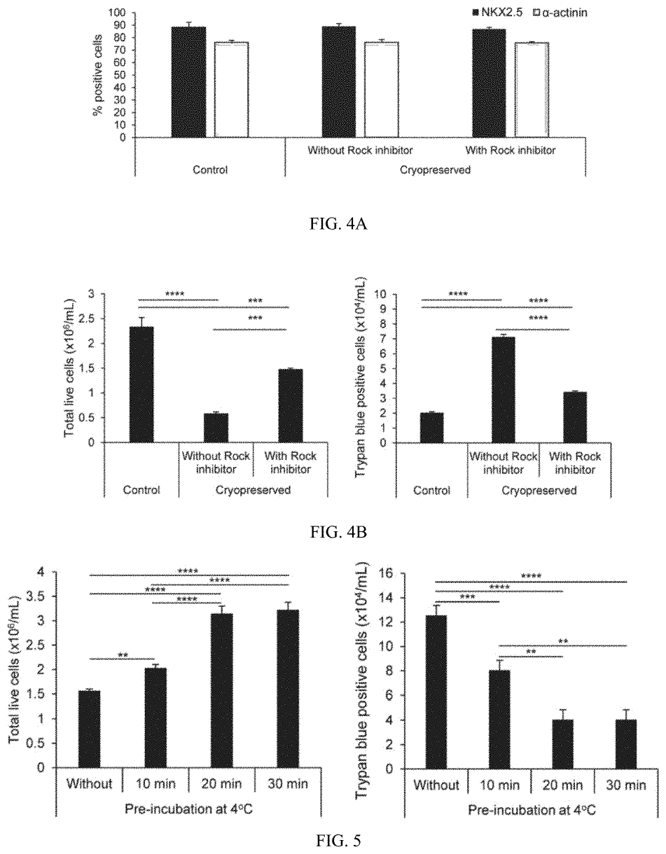

Experiments were performed to determine if the CO₂-independent medium affected mitochondrial features of hiPSC-CMs derived from cardiac progenitors maintained in the CO₂-independent medium. To examine the mitochondrial content, immunocytochemistry was performed using antibodies against Tom20, a mitochondrial marker. As analyzed by high-content imaging, hiPSC-CMs cultures in the CO₂-independent medium had slightly higher levels of mean fluorescence intensity of Tom20 compared with the parallel cultures in RPMI/B27 medium (FIG. 3A). Also examined was the mitochondrial membrane potential by TMRM, a cell-permeant dye that accumulates in active mitochondria with intact membrane potentials. As analyzed by high-content imaging, hiPSC-CMs cultures in the CO₂-independent medium had 33.1% increased levels of mean fluorescence intensity of TMRM compared with the parallel cultures in RPMI/B27 medium (FIG. 3B).

In addition, experiments were performed to determine whether the CO₂-independent medium affected the expression of genes associated with mitochondrial function. The genes examined include OPA1 which encodes a mitochondrial protein (OPA1 mitochondrial dynamin like GTPase) that localizes to the inner mitochondrial membrane and helps regulate mitochondrial stability and energy output; MFN2 (mitofusin 2) which encodes a mitochondrial membrane protein that participates in mitochondrial fusion and contributes to the maintenance and operation of the mitochondrial network; COQ10A (Coenzyme Q10A) which is required for the function of coenzyme Q in the respiratory chain; and NDUFB5 (NADH:ubiquinone oxidoreductase subunit B5). Among these genes, all had higher levels of expression in hiPSC-CMs cultured in the CO₂-independent medium than in the RPMI/B27 medium (FIG. 3C). These results suggest that hiPSC-CMs in the CO₂-independent medium may have improved mitochondrial properties, which might be contributed by medium supplements that are reported to facilitate the metabolic and functional maturation of hiPSC-CMs.

Cryopreserved Cardiac Progenitors from Hipscs Efficiently Differentiate into Cardiomyocytes

To facilitate our ground-based hardware testing and space experiments, experiments were performed to identify an effective cryopreservation method for hiPSC-derived cardiac progenitors. The objective is to allow enough time to characterize the quality of cardiac progenitors before a particular batch of progenitors is sent to the ISS or for other ground-based experiments. Sending cryopreserved cells to the ISS also allows flexible timing to start the experiment at the ISS and overcome possible negative impact on the cells that may be caused by high g-force during launching.

Experiments were performed to determine whether cardiac progenitors can be efficiently cryopreserved with high post-thawing viability and high efficiency of cardiomyocyte differentiation. Cardiac progenitors from 2D cultures on differentiation day 4 were cryopreserved at 5×10⁶ cells/vial in a cryopreservation medium containing 10% DMSO. After cell thawing and culturing in the standard culture condition, cell survival and cardiomyocyte differentiation efficiency were compared with parallel continuous cultures without cryopreservation (control). After thawing, the cryopreserved cardiac progenitors attached well to Matrigel-coated plates. Beating cells were detected on differentiation day 9 in cultures derived from control cells and cryopreserved cells. On differentiation day 15, cells in cultures without cryopreservation appeared densely packed with debris throughout the well but cultures from cryopreserved cells had visibly less debris. Similarly, flow cytometry detected 73.5% viable cells (EMA negative cells) in cryopreserved cultures and 50.9% viable cells in cultures without cryopreservation (control). The lower cell viability in cultures without cryopreservation might be due to the high density after long-term culture. The purity of cardiomyocytes (α-actinin positive cells) in cultures with and without cryopreservation was comparable.

### Rock Inhibitor Improved Cryopreservation of Intact 3D Cardiac Progenitors

Experiments were performed to determine whether 3D cardiac progenitor spheres could be cryopreserved and thawed directly in suspension culture and further differentiate into cardiomyocytes and if cell survival and differentiation of 3D cardiac progenitors could be improved with Y-27632, an inhibitor of Rho-associated, coiled-coil containing protein kinase (Rock). Cardiac progenitor spheres were cryopreserved and thawed into suspension culture in the CO₂-independent medium with or without the Rock inhibitor in cryoprotectant and thawing medium. In the following days, the culture from cryopreserved spheres without the Rock inhibitor contained more cell debris with sphere sizes that were smaller than those in the culture without cryopreservation. By contrast, the sphere morphology appeared comparable throughout the course of differentiation in cryopreserved spheres with the Rock inhibitor compared with those in cultures without cryopreservation. After 1 week in culture, the cell number and purity were compared with parallel culture without cryopreservation. ArrayScan™ analysis showed the cardiomyocyte purity was comparable among the culture conditions (˜86-89% NKX2.5 positive cells; ˜76% α-actinin positive cells). However, the total viable cell count was ˜2.5-fold higher in the cryopreserved culture with the Rock inhibitor than in the cryopreserved culture without the Rock inhibitor. These results suggest that cryopreserved intact cardiac progenitor spheres survived and differentiated following thawing and that the Rock inhibitor improved the survival of cryopreserved 3D cardiac progenitors.

### The Effect of Cell Seeding Density for Sphere Formation on Cryopreservation of 3D Cardiac Progenitors

To evaluate the effect of cell seeding density for sphere formation on cryopreservation of cardiac progenitor spheres, spheres were generated with various amounts of cells plated into Aggrewell™ 400 plates ranging from 1000 cells/sphere to 2000 cells/sphere. Frozen cardiac progenitor spheres from these preparations were then cultured in the CO₂-independent medium for 4 days. To examine cell viability, LIVE/DEAD staining was performed with calcein AM and ethidium. Cultures with a higher cell seeding density showed slightly more ethidium-positive cells than cultures with lower seeding density. Three days after thawing, spheres in all cultures had similar size. Further differentiation of these thawed cardiac progenitor spheres resulted in cardiomyocytes with similar differentiation efficiency: more than 90% cells were positive for NKX2.5 in all the cultures. These results suggest that cryopreservation outcomes are comparable with cardiac spheres generated from 1000 to 2000 cells/sphere.

Pre-Incubation with Cryopreservation Medium Improved Cell Survival of Cryopreserved 3D Cardiac Progenitors

Experiments were performed to determine whether pre-incubation of cardiac progenitor spheres with cryopreservation medium prior to freezing could improve cell survival. Cardiac spheres were collected and resuspended in freezing medium and either immediately cryopreserved at −80° C. (control) or pre-incubated for various durations at 4° C. before being cryopreserved at −80° C. Cardiac spheres were then thawed at 37° C. for 5 min and transferred into low adhesion dishes in the CO₂-independent medium with 10 μM Rock inhibitor Y-27632 and cultivated for 7 days. Cardiac spheres were then dissociated, and cell viability was tested by Trypan blue staining. A substantial increase in the total viable cells from cardiac spheres was observed when pre-incubated for 10 min (FIG. 5) compared with the control without pre-incubation. Increasing the pre-incubation time to 20-30 min resulted in even higher survival rate of the frozen cultures. These results suggest that pre-incubation with cryopreservation medium improved cell survival of cryopreserved 3D cardiac progenitors.

Serum Supplement in CO₂-Independent medium Improved Post-Thawing Cell Survival and Differentiation of 3D Cardiac Progenitors and Supported Differentiation of Functional Cardiomyocytes

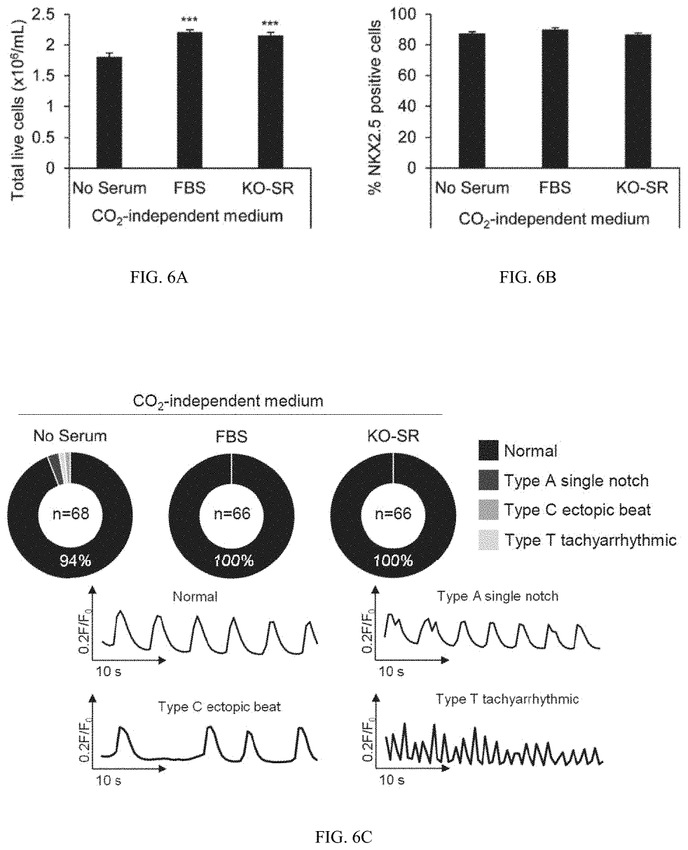

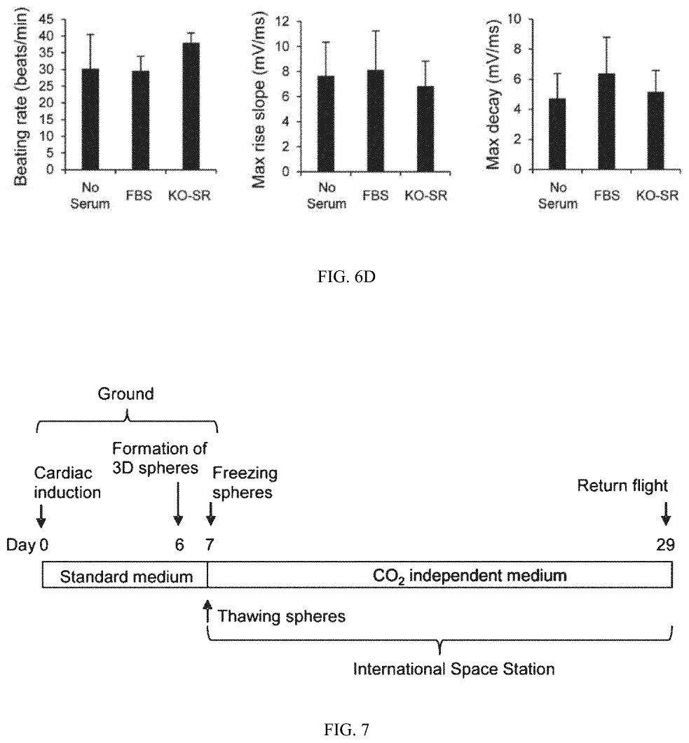

Experiments were performed to determine whether cell viability post-thawing of cardiac progenitor spheres could be further improved by adding 3% of FBS or Knockout serum replacement (KO-SR) to the CO₂-independent medium. Cryopreserved 3D cardiac progenitors were thawed and cultured in the CO₂-independent medium with or without serum supplements. Beating cardiomyocytes were observed as early as days 9-10 in all culture conditions. The total viable cells showed an improvement with the serum-supplemented formulations compared with the CO₂-independent medium without serum supplements (FIG. 6A). The purity of the cardiomyocytes based on NKX2.5 staining was ˜85% in all the culture conditions (FIG. 6B).

To evaluate the function of hiPSC-CMs following cryopreservation and culture in the CO₂-independent medium, Ca2+ transients were monitored by calcium imaging using ImageXpress™ Micro XLS System. As shown in FIG. 6C, 94% of the cells in the serum-free CO₂-independent medium had regular Ca2+ transients and all cells examined in the CO₂-independent medium with FBS or KO-SR had normal Ca2+ transients. Abnormal Ca2+ transients including peaks with single notch, ectopic beat or tachyarrhythmia were found in 6% of the cells in the serum-free CO₂-independent medium, which is within the range typically observed in hiPSC-CMs cultured in the standard medium of RPMI/B27. Furthermore, analysis of Ca2+ transient parameters indicated that cells had similar beating rate, maximum rise slope and maximum decay slope among these cultures (FIG. 6D), which are also within the range typically observed in hiPSC-CMs cultured in the standard medium of RPMI/B27.

Overall, these results indicate that 3D cardiac progenitors are able to survive post-thawing and further differentiate into functional beating cardiomyocytes in the CO₂-independent medium with serum or a defined serum replacement.

Recovery of Live Cells Following Cell Culture of Cryopreserved 3D Cardiac Progenitors without Co₂ on the International Space Station

For the spaceflight experiment, 3D cardiac progenitors were generated using Aggrewell™ 400 plates on differentiation day 6 and cryopreserved them on day 7. These samples were flown to the ISS through the SpaceX-20 mission. The astronauts on board the ISS thawed and cultured the cardiac progenitor spheres at 37° C. with the final formulation of the CO₂-independent medium. Live cultures were returned to the ground via warm storage after being cultured for 22 days on the ISS. Cell morphology and viability of the returned samples were examined. The cardiac spheres had robust beating activity and the majority of the cells in spheres were alive (positive for the green calcein AM staining), with minimal staining for ethidium (red). These results demonstrate the feasibility of cryopreservation and CO₂-independent culture of 3D cardiac progenitors for spaceflight experiments.

1. A cell growth medium pH buffered with mono sodium phosphate, dibasic sodium phosphate, beta-glycerophosphate, and sodium bicarbonate further comprising
a) insulin and B27 supplements such as biotin, alpha-tocopherol and alpha-tocopherol acetate ester, and vitamin A;
b) 4-(2-hydroxyethyl)-1-piperazineethanesulfonic acid (HEPES);
c) alanyl-glutamine dipeptide;
d) ascorbic acid, ascorbic acid 2-phosphate, or salts thereof; and
e) essential amino acids and non-essential amino acids.

2. The cell growth medium of claim 1 further comprising a saccharide or polysaccharide and pyruvate or salts or esters thereof.

3. The cell growth medium of claim 1, wherein the vitamin A is retinyl acetate.

4. The cell growth medium of claim 1, wherein the B27 supplements include catalase, transferrin, superoxide dismutase, corticosterone, galactose, ethanolamine, reduced glutathione, carnitine, linoleic acid, linolenic acid, progesterone, putrescine, selenium, triiodothyronine, or salts thereof.

5. The cell growth medium of claim 1 further comprising a serum supplement.

6. The cell growth medium of claim 1 further comprising a eukaryotic cell.

7. The cell growth medium of claim 1 further comprising cardiac cells or cardiac progenitor cells derived from induced pluripotent stem cells.

8. The cell growth medium of claim 7 further comprising an inhibitor of Rho-associated, coiled-coil containing protein kinase (Rock).

9. The cell growth medium of claim 8, wherein the inhibitor of Rho-associated, coiled-coil containing protein kinase (Rock) is 4-[1-aminoethyl]-N-4-pyridinylcyclohexanecarboxamide (Y-27632).

10. A method of culturing cells comprising contacting cells with a cell growth medium of claim 1 providing replicated cells.

11. The method of claim 10 wherein the cells are cultured in the absence of carbon dioxide.

12. The method of claim 10, wherein the cells are cardiac cells or cardiac progenitor cells derived from induced pluripotent stem cells.

13. The method of claim 12, wherein the cardiac cells or cardiac progenitor cells have a round or spherical shape.

14. The method of claim 12, wherein the cardiac cells or cardiac progenitor cells are in a planar shape.

15. A method of preserving and culturing cells in outer space comprising;
a) freezing cells providing frozen cells;
b) transporting the frozen cells to outer space;
c) thawing the cells providing thawed cells; and
d) culturing the thawed cells with a growth medium of claim 1.

16. The method of claim 15, wherein freezing cells is in a medium contain dimethyl sulfoxide.

17. The method of claim 15, wherein the cells are cardiac progenitor cells rounded or spheroidal in shape suspended in the growth medium.

18. The method of claim 15, wherein the cells are pre-incubated for a duration at 4° C. before being frozen.

19. The method of claim 18, wherein the duration is for at least 20 minutes.

20. The method of claim 15, wherein the cells are cultured in the absence of atmospheric carbon dioxide for more than 10 days.
